SEQUENCE LISTING (10 sequences)

SEQ ID NO: 1 - DNA, 39 bp, Artificial Sequence
  Synthetic Polynucleotide
aatgggaaaaagttaacaaaatgtacaaggaccgacaag

SEQ ID NO: 2 - DNA, 39 bp, Artificial Sequence
  Synthetic Polynucleotide
ggtaggtaacacatatatttttcttgatacttgcagaaa

SEQ ID NO: 3 - DNA, 39 bp, Artificial Sequence
  Synthetic Polynucleotide
cttgtcggtccttgtacattttgttaactttttcccatt

SEQ ID NO: 4 - DNA, 39 bp, Artificial Sequence
  Synthetic Polynucleotide
tttctgcaagtatcaagaaaaatatatgtgttacctacc

SEQ ID NO: 5 - DNA, 34 bp, Artificial Sequence
  Synthetic Polynucleotide
gtaacacatatatttttcttgatacttgcagaaa

SEQ ID NO: 6 - DNA, 34 bp, Artificial Sequence
  Synthetic Polynucleotide
cggtccttgtacattttgttaactttttcccatt

SEQ ID NO: 7 - DNA, 14 bp, Artificial Sequence
  Synthetic Polynucleotide
acgtaccgtacggt

SEQ ID NO: 8 - DNA, 14 bp, Artificial Sequence
  Synthetic Polynucleotide
accgtacggtacgt

SEQ ID NO: 9 - DNA, 13 bp, Artificial Sequence
  Synthetic Polynucleotide
acgtacgtacggt

SEQ ID NO: 10 - DNA, 13 bp, Artificial Sequence
  Synthetic Polynucleotide
accgtacgtacgt

(19) United States
(12) Patent Application Publication
(10) Pub. No.: US 2020/0040329 A1
(43) Pub. Date: Feb. 6, 2020

(54) SYSTEMS AND METHODS FOR PREDICTING REPAIR OUTCOMES IN GENETIC ENGINEERING

(71) Applicant: Massachusetts Institute of Technology, Cambridge, MA (US)

(72) Inventors: David K. Gifford, Boston, MA (US); Max Walt Shen, Cambridge, MA (US); Jonathan Yee-Ting Hsu, Cambridge, MA (US)

(21) Appl. No.: 16/538,408

(22) Filed: Aug. 12, 2019

Related U.S. Application Data
(63) Continuation of application No. PCT/US2018/065885, filed on Dec. 15, 2018.
(60) Provisional application No. 62/599,623, filed on Dec. 15, 2017.

(51) Int. Cl. C12N 15/10; C12N 15/11; C12N 9/22; G16B 40/00; G16B 20/20; G16B 5/20; G06N 3/08; G06N 3/04

(52) U.S. Cl. CPC C12N 15/1089; C12N 15/11; C12N 9/22; G16B 40/00; C12N 2310/20; G16B 5/20; G06N 3/08; G06N 3/04; G16B 20/20

(57) ABSTRACT

The specification provides a machine-learning model which predicts, based on input that can include a given target DNA sequence and a CRISPR/Cas cut site location, repair genotype outcomes associated with template-free repair processes (e.g., MMEJ or NHEJ) acting on Cas9-induced double-stranded DNA breaks. The specification further provides for the use of a machine-learning model for conducting genome editing based on a template-free CRISPR/Cas system, including the selection of an appropriate guide RNA (gRNA) to achieve a desired repaired genotype outcome.

### RELATED APPLICATIONS

This application is a continuation of PCT/US2018/065885, filed Dec. 15, 2018, which claims the benefit of and priority to U.S. Provisional Patent Application No. 62/599,623, filed on Dec. 15, 2017, entitled “SYSTEMS AND METHODS FOR PREDICTING REPAIR OUTCOMES IN GENETIC ENGINEERING,” which are incorporated herein by reference in their entirety.

### FEDERALLY SPONSORED RESEARCH

This invention was made with Government support under Grant No. R01 HG008754 awarded by the National Institute of Health. The government has certain rights in the invention.

### REFERENCE TO SEQUENCE LISTING

The Sequence Listing submitted Aug. 12, 2019, as a text file named “MIT_21045_ST25.txt,” created on Aug. 12, 2019, and having a size of 2,297 bytes is hereby incorporated by reference pursuant to 37 C.F.R. § 1.52(e)(5).

### BACKGROUND

CRISPR/Cas9 has revolutionized genome editing and engineering, providing an invaluable research tool in genetics and emerging as a promising tool for genetic treatment of disease. CRISPR creates a double-strand break at a user-specified location in a genome. This double-strand break is repaired by cells through a plurality of DNA repair pathways, resulting in a plurality of repair genotypes with varying frequencies.

One DNA repair pathway, homology-directed repair (HDR), enables incorporation of a user-designed DNA sequence into a genome. This is accomplished by introducing into a cell a homologous DNA repair template comprising the desired DNA sequence. Other repair pathways, including non-homologous end-joining (NHEJ) and microhomology-mediated end-joining (MMEJ), occur without any template, and usually result in nucleotide insertions and/or deletions (sometimes referred to as “indels”). It has generally been the accepted view that template-free repair processes result in a random distribution of genome outcomes. Further, prior to the disclosure that follows, there have been no general methods described which accurately predict repair genotype outcomes associated with template-free repair processes acting on Cas9-induced double-stranded DNA breaks.

### SUMMARY

Aspects of the technology disclosed herein relate to a machine-learning model which predicts, based on input that can include a given target DNA sequence and a CRISPR/Cas cut site location, repair genotype outcomes associated with template-free repair processes (e.g., MMEJ or NHEJ) acting on Cas9-induced double-stranded DNA breaks. The disclosed invention further relates to the use of the machine-learning computational model for conducting genotypic editing (e.g., genotypic correction of a pathogenic allele) based on template-free CRISPR/Cas editing, including the selection of an appropriate guide RNA (gRNA) to achieve the desired repaired genotype outcome.

In one aspect, the invention provides a method for selecting one or more guide RNAs (gRNAs) from a plurality of gRNAs for CRISPR, comprising acts of:

for at least one gRNA of the plurality of gRNAs, using a local DNA sequence and a cut site targeted by the at least one gRNA to predict a frequency of one or more repair genotypes resulting from template-free repair following application of CRISPR with the at least one gRNA; and

determining whether to select the at least one gRNA based at least in part on the predicted frequency of the one or more repair genotypes.

In certain embodiments, that one or more repair genotypes correspond to one or more healthy alleles of a gene related to a disease.

In other embodiments, the predicted frequency of the one or more repair genotypes is at least about 50%.

In various embodiments, the step of predicting the frequency of the one or more repair genotypes comprises:

for each deletion length of a plurality of deletion lengths, aligning subsequences of that deletion length on 5′ and 3′ sides of the cut site to identify one or more longest microhomologies;

featurizing the identified microhomologies;

applying a machine learning model to compute a frequency distribution over the plurality of deletion lengths, wherein the computation includes a non-linear function of the number of matches in said microhomologies; and using the frequency distribution over the plurality of deletion lengths to determine the frequency of the one or more repair genotypes.

In certain embodiments, the step of featurizing the identified microhomologies comprises determining a G-C fraction value for each of the identified microhomologies.

In certain other embodiments, the step of featurizing the identified microhomologies further comprises determining a microhomology length of each of the identified microhomologies.

In still other embodiments, applying the machine learning model comprises applying a neural network model.

In other embodiments, the step of predicting the frequency of the one or more repair genotypes comprises:

for each deletion length of a plurality of deletion lengths, aligning subsequences of that deletion length on 5′ and 3′ sides of the cut site to identify one or more longest microhomologies; determining feature values for the identified microhomologies; and

providing the feature values as input to a machine learning model to obtain output indicating a probability distribution over a plurality of deletion lengths.

In yet other embodiments, the step of predicting the frequency of the one or more repair genotypes further comprises:

using the probability distribution over the plurality of deletion lengths to determine the frequency of the one or more repair genotypes.

In still other embodiments, that plurality of gRNAs comprise gRNAs for CRISPR/Cas9, and the application of CRISPR comprises application of CRISPR/Cas9.

In another aspect, the invention provides a system comprising:

at least one processor; and

at least one computer-readable storage medium having encoded thereon instructions which, when executed, cause the at least one processor to perform any of the above methods.

In various embodiments, the computer-readable storage medium having encoded thereon instructions which, when executed, causes at least one processor to perform any of the above methods.

The invention, in another aspect, provides for CRISPR editing of DNA that utilizes a guide RNA in the absence of a homology directed repair template, wherein the guide RNA is selected to produce one or more selected genotypic outcomes.

In still another aspect, the invention provides a method of predicting a frequency of one or more repair genotypes resulting from template-free repair following application of template-free CRISPR/Cas to a target nucleotide sequence, the method comprising:

using at least one computer hardware processor to perform:

for each deletion length of a plurality of deletion lengths, aligning subsequences of that deletion length on 5′ and 3′ sides of a cut site to identify one or more longest microhomologies;

determining feature values for the identified microhomologies;

providing the feature values as input to a machine learning model to obtain output indicating a probability distribution over the plurality of deletion lengths; and

using the probability distribution over the plurality of deletion lengths to determine the frequency of the one or more repair genotypes.

In various embodiments, the step of determining the feature values comprises: determining a G-C fraction value for each of the identified microhomologies.

In still other aspects, the step of determining the feature values comprises:

determining a microhomology length of each of the identified microhomologies.

In various embodiments, the machine learning model comprises a neural network model, which can comprise multiple hidden layers (e.g., 2, 4, 5, 6, or more hidden layers).

In other embodiments, for each deletion length of the plurality of deletion lengths, the method can comprising the further step of aligning subsequences of that deletion length on 5′ and 3′ sides of a cut site to identify two or more longest microhomologies.

In another aspect, the present invention provides a system comprising:

at least one processor; and

at least one computer-readable storage medium having encoded thereon instructions which, when executed, cause the at least one processor to perform any of the above methods.

In another aspect, the invention further relates to at least one computer-readable storage medium having encoded thereon instructions which, when executed, cause at least one processor to perform any of the above methods.

### BRIEF DESCRIPTION OF DRAWINGS

FIG. 1 shows an illustrative DNA segment 100, in accordance with some embodiments. The sequences are: top left (SEQ ID NO:1), top right (SEQ ID NO:2), bottom left (SEQ ID NO:3), and bottom right (SEQ ID NOs:4).

FIGS. 2A-D show an illustrative matching of 3′ ends of top and bottom strands of a DNA segment at a cut site and an illustrative repair product, in accordance with some embodiments. The sequences in FIG. 2A are: top left (SEQ ID NO:1), top right (SEQ ID NO:5), bottom left (SEQ ID NO:6), and bottom right (SEQ ID NOs:4).

FIG. 2E depicts the general mechanism for generating a CRISPR-mediated double strand DNA break, and the MMEJ repair pathway for repairing the double strand DNA break.

FIG. 3 shows an illustrative process 300 for building a machine learning model for predicting frequencies of deletion lengths, in accordance with some embodiments.

FIG. 4 shows an illustrative neural network 400 for computing a frequency distribution over deletion lengths, in accordance with some embodiments.

FIG. 5 shows an illustrative process 500 for processing data collected from CRISPR/Cas experiments, in accordance with some embodiments.

FIG. 6 shows an illustrative process 600 for using a machine learning model to predict frequencies of deletion lengths, in accordance with some embodiments.

FIG. 7 shows illustrative examples of a blunt-end cut and a staggered cut, in accordance with some embodiments. The sequences are, from top to bottom, SEQ ID NOs:9, 10, 9, 10, 7, 8, 7, and 9.

FIG. 8 shows, schematically, an illustrative computer 1000 on which any aspect of the present disclosure may be implemented.

FIG. 9A shows the experimentally observed deletion length distribution of a first target sequence and cut site. FIG. 9B shows the corresponding predicted distribution of deletion lengths for the same target sequence and cut site as calculated by the neural network 400.

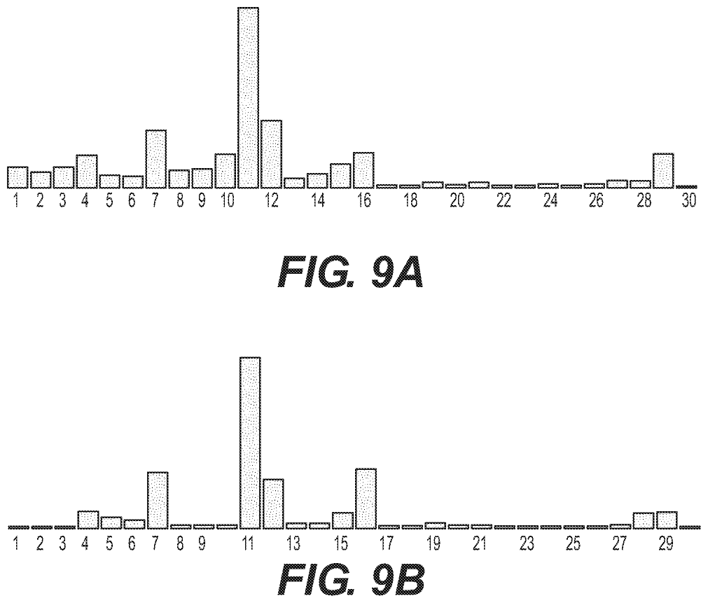

FIG. 9C shows the experimentally observed deletion length distribution of a second target sequence and cut site. FIG. 9D shows the corresponding predicted distribution of deletion lengths for the same target sequence and cut site as calculated by the neural network 400.

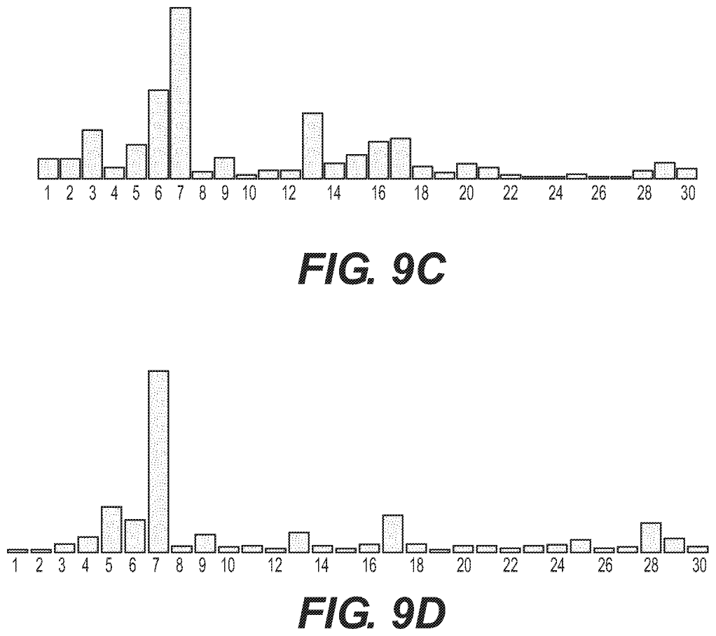

### DETAILED DESCRIPTION

Major research efforts focus on improving efficiency and specificity of CRISPR/Cas9 DNA cutting. For instance, efficiency may be improved by predicting optimal Cas9 guide RNA (gRNA) sequences, while specificity may be improved by modeling factors leading to off-target cutting, and by manipulating Cas9 enzymes. Variant Cas9 enzymes and fusion proteins may be developed to alter the protospacer adjacent motif (PAM) sequences acted on by Cas9, and to produce base-editing Cas9 constructs with high efficiency and specificity. For example, Cpf1 (also known as Cas12a) and other alternatives may be used in CRISPR genome editing in addition to, or instead of, Cas9.

The inventors have recognized and appreciated that less attention has been devoted to understanding and modulating repair outcomes. In that respect, nucleotide insertions and/or deletions resulting from template-free repair mechanisms (e.g., NHEJ, MMEJ, etc.) are commonly thought to be random and therefore only suitable for gene knock-out applications. For gene knock-in or gain-of-function applications, a template-based repair mechanism such as HDR is typically used.

CRISPR/Cas with HDR allows arbitrarily designed DNA sequences to be incorporated at precise genomic locations. However, this technique suffers from low efficiency—HDR occurs rarely in typical biological conditions (e.g., around 10% frequency), because cells typically only permit HDR to occur after sister chromatids are synthesized in S phase but before M phase when mitosis splits the sister chromatids into daughter cells. For many cell-types, the fraction of time spent in S-G2-M phases of a cell cycle is low. In sum, while outcomes are predictable when HDR does occur, HDR occurs infrequently, and therefore a desired DNA sequence will be incorporated into only a small percentage of cells. In addition, in post-mitotic cell-types of interest such as neurons, the HDR repair pathway is no longer used, further limiting HDR's utility for genetic engineering.

Some research has been done to improve efficiency of HDR, for example, through improved homology templates and small molecule modulation. Despite these efforts, template-based repair efficiency remains low, and proposed CRISPR/Cas gene knock-in or gain of function applications have thus far been limited to ex vivo applications where screening may be performed for cells with a desired repair genotype.

Unlike HDR, NHEJ is capable of occurring during any phase of a cell cycle and in post-mitotic cells. However, NHEJ, as discussed above, has been perceived as a random process that produces a large variety of repair genotypes with insertions and/or deletions, and has been used mainly to knock out genes. In short, NHEJ is efficient but unpredictable.

Recent work suggests that outcomes of some template-free repair mechanisms are actually non-random. For instance, it has been observed that MMEJ is involved in repair outcomes. Furthermore, repair outcomes have been analyzed to predict gRNAs that are more likely to produce frameshifts. However, there is still a need for accurate prediction of genotypic outcomes of CRISPR/Cas cutting and ensuing cellular DNA repair.

In accordance with some embodiments, techniques are provided for predicting genotypes of CRISPR/Cas editing outcomes. For instance, a high-throughput approach may be used for monitoring CRISPR/Cas cutting outcomes, and/or a computer-implemented method may be used to predict genotypic repair outcomes for NHEJ and/or MMEJ. The inventors have recognized and appreciated that accurate prediction of repair genotypes may allow development of CRISPR/Cas gene knock-in or gain-of-function applications based on one or more template-free repair mechanisms. This approach may simplify a genome editing process, by reducing or eliminating a need to introduce exogenous DNA into a cell as a template.

Additionally, or alternatively, using one or more template-free repair mechanisms for gene knock-in may provide improved efficiency. For instance, the inventors have recognized and appreciated that NHEJ and MMEJ may account for a large portion of CRISPR/Cas repair products. While template-free repair mechanisms may not always produce desired repair genotypes with sufficiently high frequencies, one or more desired repair genotypes may occur with sufficiently high frequencies in some specific local sequence contexts. For such a local sequence context, template-free repair mechanisms may outperform HDR with respect to simplicity and efficiency.

In some embodiments, one or more of the techniques provided herein may be used to predict, using a machine learning model and for a given local sequence context, template-free repair genotypes and frequencies of occurrence thereof, which may facilitate designs of gene knock-in or gain-of-function applications. For example, the inventors have recognized and appreciated that some disease-causing alleles, when cut at a selected location by CRISPR/Cas, may exhibit one or just a few repair outcomes that occur at a high frequency and transform the disease-causing allele into one or more healthy alleles. Disease-causing alleles may occur in genomic sequences that code for proteins or regulatory RNAs, or genomic sequences that regulate transcription or other genomic functions.

It should be appreciated that the techniques disclosed herein may be implemented in any of numerous ways, as the disclosed techniques are not limited to any particular manner of implementation. Examples of details of implementation are provided solely for illustrative purposes. For instance, while examples are given where CRISPR/Cas9 is used to perform genome editing, it should be appreciated that aspects of the present application are not so limited. In some embodiments, another genome editing technique, such as CRISPR/Cpf1, may be used. Furthermore, the disclosed techniques may be used individually or in any suitable combination, as aspects of the present disclosure are not limited to the use of any particular technique or combination of techniques.

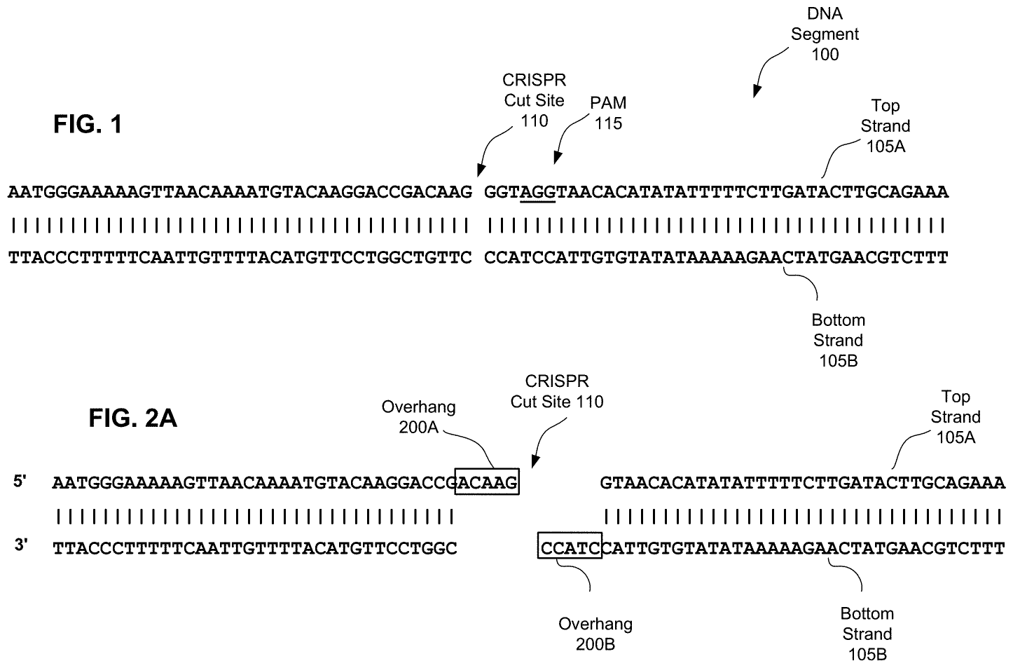

FIG. 1 shows an illustrative DNA segment 100, in accordance with some embodiments. For instance, the DNA segment 100 may be exon 43 of a dystrophin gene. About 4% of Duchenne's muscular dystrophy cases are caused by mutations in this exon. Therapeutic solutions showing success in clinical trials use antisense oligonucleotides to cause this exon to be skipped during translation, thereby restoring normal dystrophin function.

The inventors have recognized and appreciated that another therapeutic approach may be possible, using genome editing to make permanent changes to dystrophin exon 43. For instance, in some embodiments, CRISPR/Cas9 (or another suitable technique for cutting a DNA sequence, such as CRISPR/Cpf1) may be used to disrupt a donor splice site motif of dystrophin exon 43, and one or more template-free repair mechanisms may restore normal dystrophin function.

In the example shown in FIG. 1, the DNA segment 100 includes a top strand 105A and a bottom strand 105B. These two strands are complementary and therefore encode the same information. In some embodiments, CRISPR/Cas9 may be used to create a double strand cut at a selected donor splice site motif, which may be a specific sequence of 6-10 nucleotides. In the example of FIG. 1, an NGG PAM may be used, as underlined and shown at 115, so that a cut site 110 would occur within the selected donor splice site motif. Any suitable algorithm may be used to detect presence or absence of the splice site motif in repair products, thereby verifying if the splice site motif has been successfully eliminated.

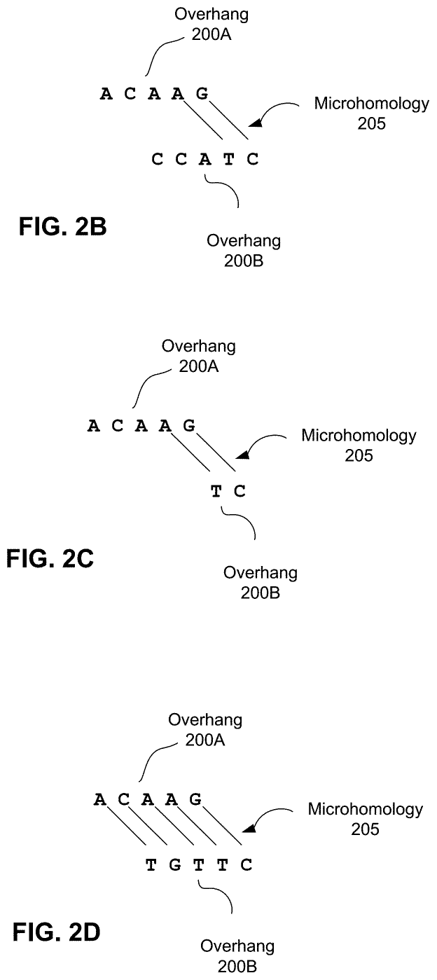

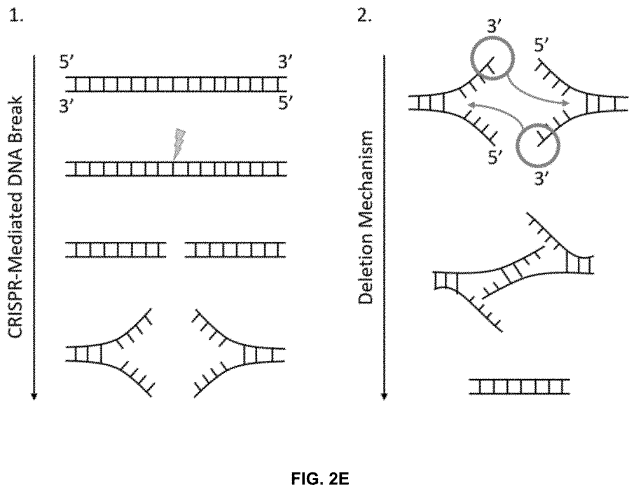

FIGS. 2A-D show an illustrative matching of 3′ ends of top and bottom strands of a DNA segment at a cut site and an illustrative repair product, in accordance with some embodiments. For instance, the strands may be the illustrative top strand 105A and the illustrative bottom strand 105B of FIG. 1, and the cut site may be the illustrative cut site 110 of FIG. 1. (To avoid clutter, the surrounding sequence context is omitted in FIGS. 2B-D.)

In some embodiments, a segment of double-stranded DNA may be represented such that the top strand runs 5′ on the left to 3′ on the right. Given a cut in this double stranded DNA, nucleotides and their complementary base-paired nucleotides that lie between the 5′ end of the top strand and the cut site may be said to be located at the 5′ side of the cut site. Likewise, nucleotides and their complementary base-paired nucleotides that lie between the cut site and the 3′ end of the top strand may be said to be located at the 3′ side of the cut site.

In the example shown in FIG. 2A, a deletion length of 5 base pairs is considered, for example, as a result of 5′ end resection, where the top strand 105A has an overhang 200A of length 5 at the 5′ side of the cut site 110, and the bottom strand 105B has an overhang 200B of length 5 at the 3′ side of the cut site 110. As shown in FIG. 2B, there is no match between the overhangs 200A and 200B in the first three bases, but there is a match in each of the last two bases. Thus, in this example, a microhomology 205 is present, with a 2 base pair match.

FIG. 2C shows an illustrative result of flap removal, where the three mismatched bases in the overhang 200B are removed. For instance, in some embodiments, given a microhomology, some or all nucleotides on the 3′ side of the microhomology on the top strand, and/or some or all nucleotides on the 3′ side of the microhomology on the bottom strand, may be resected. Pictorially, with the top strand running 5′ to 3′, nucleotides to the right of the microhomology on the top strand may be resected, and nucleotides to the left of the microhomology on the bottom strand may be resected.

FIG. 2D shows an illustrative repair product resulting from polymerase fill-in and ligation, where three matching bases are added to the overhang 200B.

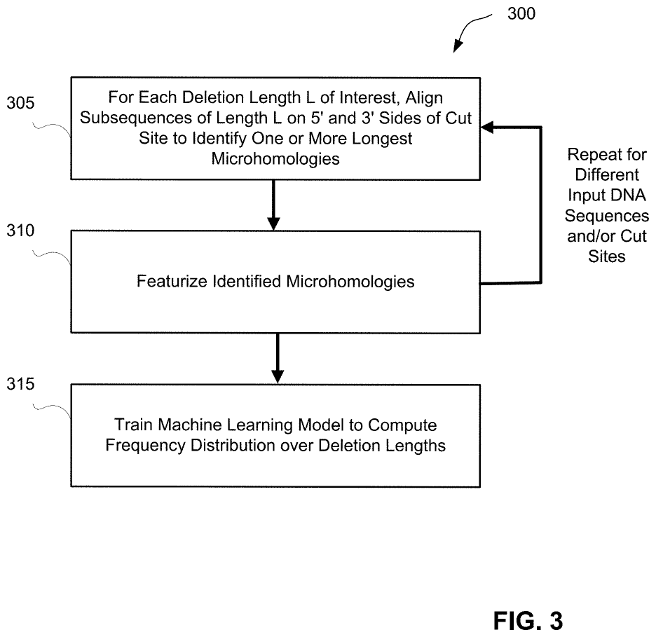

FIG. 3 shows an illustrative process 300 for building a machine learning model for predicting frequencies of deletion lengths, in accordance with some embodiments. In some embodiments, the machine learning model may be a neural network (e.g., a multi-layer neural network which is sometimes termed a “deep” neural network, a fully-connected neural network), a linear regression model, a non-linear regression model, an adaptive regression model, a random forest regression model, a support vector machine, a statistical model, a graphical model, a Bayesian model, and/or any other suitable type of machine learning model, as aspects of the technology herein are not limited in this respect.

In some embodiments, building the machine learning model may include training the machine learning model using training data. The training data may include input-output pairs, in some embodiments. In some embodiments, the machine learning model may include parameters, and training the machine learning model using training data may include using the training data to estimate values of one or more (e.g., all) of the parameters. Additionally, in some embodiments, the machine learning model may include one or more hyper-parameters (e.g., the number of nodes in a hidden layer of a neural network, the number of layers in a neural network, the non-linearity associated with one or more nodes in the neural network, the topology of the neural network, etc.), and training the machine learning model using training data may include estimating values of one or more (e.g., all) of the hyper-parameters using the training data.

In some embodiments, the process 300 may be used to build a machine learning model that computes, given an input DNA sequence seq and a cut site location, a probability distribution over any suitable set of deletion lengths. In some embodiments, a probability distribution over deletion lengths from 3 to 26 may be computed. For instance, some research suggests that deletion lengths of 1-2 may result primarily from NHEJ, whereas deletion lengths of 3 and greater may result primarily from MMEJ. Therefore, in some embodiments, different prediction techniques may be used for different deletion lengths, where a prediction technique may be chosen based on one or more known behaviors of a likely repair mechanism for one or more deletion lengths of interest. For example, NHEJ may exhibit more randomness than MMEJ, and a prediction technique designed for MMEJ may be applied to deletion lengths of 3 and greater. In some embodiments, an upper limit of deletion lengths may be determined based on availability of data and/or any other one or more suitable considerations.

In some embodiments, an input DNA sequence seq may be represented as a vector with integer indices, where each element of the vector is a nucleotide from the set,

{A, C, G, T}, and the cut site is between seq[−1] and seq[0], and seq is oriented 5′ on the left to 3′ on the right. A subsequence seq[i:j], i<j, may be a vector of length j−i, including elements s[i] to s[j−1]. For each deletion length L of interest (e.g., L between 3 and 26), left[L] may be used to denote seq[−L:0], and right[L] may be used to denote seq[0, L]. Thus, with reference to the example shown in FIGS. 1, 2A, left[5] may be ACAAG, and right[5] may be GGTAG. Because the top strand 105A and the bottom strand 105B are complementary, a microhomology (e.g., the microhomology 205) may be identified by looking for exact matches (which may be referred to herein as “matches” or “matching bases”) between left[5] and right[5] (which may be equivalent to complementary matches between the overhang 200A and the overhang 200B). For instance, a match vector may be constructed for each deletion length L of interest (e.g., L between 3 and 26) as follows: match[L][i]=‘|’ if left[L][i]=right[L][i], otherwise match[L][i]=‘.’ Such matching between left[5] and right[5] is illustrated below.

ACAAG
...||
GGTAG

Although examples of representations of DNA sequences and subsequences are discussed herein, it should be appreciated that aspects of the present disclosure are not limited to the use of any particular representation.

Referring to FIG. 3, act 305 of the process 300 may include, for each deletion length L of interest (e.g., each deletion length between 3 and 26), aligning subsequences of length L on the 5′ and 3′ sides of a cut site in an input DNA sequence to identify one or more microhomologies. As used herein, the term “microhomologies” refers to contiguous run of matching bases. This may be performed for an input DNA sequence and a cut site for which repair genotype data from an CRISPR/Cas9 experiment is available.

In some embodiments, a microhomology may be identified by looking for match[L][i:j] such that match[L][k]=‘|’ for all i<k<j and match[L][i]!=‘|’ and match[L][j] !=‘|’. For instance, with reference to the example shown in FIG. 1, there may be no microhomology for deletion length 3, no microhomology for deletion length 4, one microhomology for deletion length 5, three microhomologies for deletion length 6, etc., as illustrated below.

AAG
...
GGT
CAAG
....
GGTA
ACAAG
...||
GGTAG
GACAAG
|..|.|
GGTAGG

The inventors have recognized and appreciated that longer microhomologies are more likely to play a role in template-free repair compared to shorter microhomologies. Accordingly, in some embodiments, one or more longest microhomologies may be identified for each deletion length of interest. For instance, two longest microhomologies may be identified for each deletion length of interest. While considering more longest microhomologies (e.g., three, four, five, etc.) may provide more accurate prediction results, more computation may be needed (e.g., to train a machine learning such as a neural network model, as discussed below). The inventors have recognized and appreciated that using two longest microhomologies may represent a desired tradeoff between accuracy and speed. The number of longest microhomologies considered may be denoted by the variable “B” and the number of deletion lengths of interest may be denoted by the variable “N.”

At act 310, the longest microhomologies identified at act 305 may be featurized. As used herein, the “featurizing” a microhomology refers to determining a value (e.g., calculating a value, accessing a previously calculated value) for each of one or more features of the microhomology. Thus, featurizing a microhomology may include determining one or multiple feature values characterizing the microhomology. Values for any suitable number of features may be calculated. In some embodiments, values of one or more of the following features may be calculated when “featurizing” a microhomology: (1) a GC fraction indicating fraction of bases in the microhomology that are G or C (an AT fraction indicating 1-GC fraction may be used additionally or alternatively); (2) the ratio of the microhomology length and the deletion length; (3) the position of the middle (and/or any other base) of the microhomology in the deletion, where 5′ is 0 and 3′ is 1; (4) the length of the microhomology. Additionally or alternatively, any other suitable feature(s) may be used, including any other feature described herein, as aspects of the present disclosure are not so limited.

As one example, the inventors have recognized and appreciated that energetic stability of a microhomology may increase proportionately with a length of the microhomology. Accordingly, in some embodiments, a microhomology length j−i may be used as a feature for a microhomology match[L][i:j].

As another example, the inventors have recognized and appreciated that thermodynamic stability of a microhomology may depend on specific base pairings, and that G-C pairings have three hydrogen bonds and therefore have higher thermodynamic stability than A-T pairings, which have two hydrogen bonds. Accordingly, in some embodiments, a GC fraction, as shown below, may be used as a feature for a microhomology match[L][i:j], where indicator(boolean) equals 1 if boolean is true, and 0 otherwise.

$$\frac{{\sum }_{k = i}^{j - 1} indicator ( top [ L ] [ k ] = ' G ' or ' C ' )}{j - i}$$

In some embodiments, a number of deletion lengths of interest may be N (e.g., 24—all deletion lengths between 3 and 26), and for each deletion length, B longest microhomologies may be considered. Thus, there may be N×B microhomologies, and an N×B matrix may be constructed for each feature (e.g., microhomology length, GC fraction, etc.).

In some embodiments, acts 305 and 310 may be repeated for different input DNA sequences and/or cut sites for which repair genotype data from CRISPR/Cas9 experiments is available.

It should be appreciated that aspects of the present disclosure are not limited to any particular featurization technique. For instance, in some embodiments, two features may be used, such as microhomology length and GC fraction. However, that is not required, as in some embodiments one feature may be used (e.g., microhomology length, GC fraction, or some other suitable feature), or more than two features may be used (e.g., three, four, five, etc.). Examples of features that may be used for a microhomology match[L][i:j] within a deletion of length L include, but are not limited to, a position of the microhomology within the deletion (e.g., as represented by

$$\frac{{\sum }_{k = i}^{j - 1} k}{L * ( j - i )} ) ,$$

and a ratio between a length of the microhomology (i.e., j−i) and the deletion length L. As another example, the inventors have recognized and appreciated that deoxyribonuclease (DNase) hypersensitivity may be used to classify genomic sequences into open or closed chromatin, which may impact DNA repair outcomes. Accordingly, in some embodiments, open vs. closed chromatin may be used as a feature. Any one or more of these features, and/or other features, may be used in addition to, or instead of, microhomology length and GC fraction. Furthermore, in some embodiments, explicit featurization may be reduced or eliminated by automatically learning data representations (e.g., using one or more deep learning techniques such as, for example, an auto-encoder).

Returning to FIG. 3, a machine learning model may be trained at act 315 to compute a frequency distribution over deletion lengths. For instance, a neural network model may be used that takes as input an N×B matrix for each of one or more features, as constructed at act 310, and outputs a frequency distribution over deletion lengths. The neural network model may then be trained using repair genotype data collected from CRISPR/Cas9 experiments. The neural network model may be trained using stochastic gradient descent, implemented via backpropagation, or in any other suitable way.

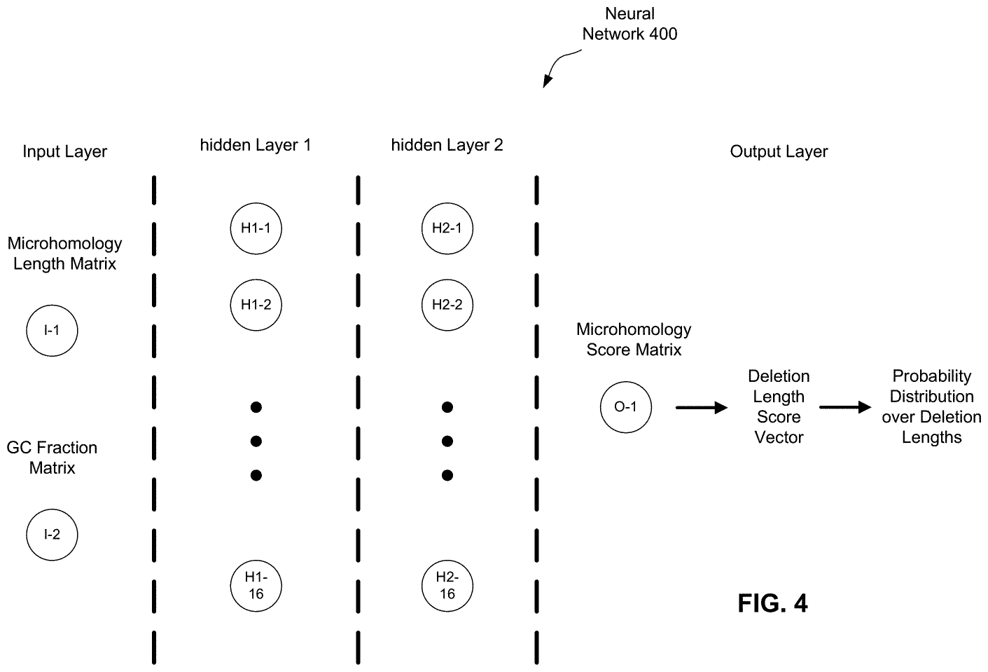

FIG. 4 shows an illustrative neural network 400 for computing a frequency distribution over deletion lengths, in accordance with some embodiments. For instance, the neural network 400 may be trained at act 315 of the illustrative process 300 shown in FIG. 3.

In some embodiments, the neural network 400 may have one input node for each microhomology feature being used. For instance, in the example shown in FIG. 4, there are two input nodes, which are associated with microhomology length and GC fraction, respectively. Each input node may receive an N×B matrix of values. In some embodiments, one or more of the positions in this matrix may have a default value. For instance, with reference to the example shown in FIG. 1, there may be no microhomology of length 3, and the corresponding feature values in the N×B matrix may be a suitable default value (e.g., 0 for microhomology length and −1 for GC fraction).

In some embodiments, the neural network 400 may include one or more hidden layers, each having one or more nodes. In the example shown in FIG. 4, there are two hidden layers, each having 16 nodes. However, it should be appreciated that aspects of the present disclosure are not limited to the use of any particular number of hidden layers or any particular number of nodes in a hidden layer. Furthermore, different hidden layers may have different numbers of nodes.

In some embodiments, the neural network 400 may be fully connected. (To avoid clutter, the connections are not illustrated in FIG. 4.) However, that is not required. For instance, in some embodiments, a dropout technique may be used, where a parameter p may be selected, and during training each node's value is independently set to 0 with probability p. This may result in a neural network that is not fully connected.

In some embodiments, a leaky rectified linear unit (ReLU) nonlinearity sigma may be used in the neural network 400. For instance, at hidden layer h and node i, an activation function may be provided as follows:

unit[h][i]=sigma(w[h][i]*unit[h−1]+b[h][i]),

where sigma(x)=max(0, x)+0.001*min(0, x). Other nonlinearities may be used, examples of which are provided herein.

Thus, in some embodiments, the neural network 400 may be parameterized by w[h] and b[h] for each hidden layer h. In some embodiments, during training, these parameters may be initialized randomly, for example, from a spherical Gaussian distribution with some suitable center (e.g., 0) and some suitable variance (e.g., 0.1). These parameters may then be trained using repair genotype data collected from CRISPR/Cas9 experiments, for instance, as discussed below.

In some embodiments, the neural network 400 may have one output node, producing an N×B matrix Z of values. Each value in this matrix may be associated with one of B longest microhomologies for deletion length L, and therefore may be referred to herein as a microhomology score.

In some embodiments, the neural network 400 may operate independently for each microhomology, taking as input the length of that microhomology (from the first input node) and the GC fraction of that microhomology (from the second input node), transforming those two values into 16 values (at the first hidden layer), then transforming those 16 values into 16 other values (at the second hidden layer), and finally outputting a single value (at the output node). In such an embodiment, parameters for the first hidden layer, w[1][i] and b[1][i], are vectors of length 2 for each node i from 1 to 16, whereas parameters for the second hidden layer, w[2][i] and b[2][i], are vectors of length 16 for each node i from 1 to 16, and parameters for the output layer, w[3][1] and b[3][1], are also vectors of length 16.

In some embodiments, the N×B matrix Z of microhomology scores from the output node may be flattened into a vector Z of N values, where each value may be associated with a deletion length L, and may be referred to herein as a deletion length score. For each deletion length L, the B microhomology scores Z[L, b] may be combined in any suitable manner For example, a weighted sum of the B microhomology scores may be computed to obtain a deletion length score. For instance, a score for the second longest microhomology may be multiplied by a weight (e.g., 0.1), and a result may be added to a score for the longest microhomology to obtain the deletion length score Z̅[L].

In some embodiments, the vector Z̅ of deletion length scores may be normalized into a probability distribution over all deletion lengths of interest (e.g., deletion lengths between 3 and 26, inclusive). The inventors have recognized and appreciated (e.g., from experimental data) that frequency may decrease exponentially with deletion length. Accordingly, in some embodiments, an exponential linear model may be used to normalize the vector of deletion length scores. For example, a softmax normalization may be included. For instance, in an example in which deletion lengths between 3 and 26 are of interest (and thus Z̅ is indexed from 3 to 26), the following formula may be used:

$$Y [ L ] = \frac{exp ( \overline{Z} [ L ] - beta * L + ci )}{{\sum }_{l = 3}^{26} exp ( \overline{Z} [ l ] - beta * l + ci )} ,$$

where L is a deletion length of interest, and beta and ci are parameters.

In some embodiments, the parameters beta and ci may be initialized to −1 and 0, respectively. These parameters may then be trained using repair genotype data collected from CRISPR/Cas9 experiments, for instance, as discussed below.

In some embodiments, the parameters w [h] and b[h] for each hidden layer h and the parameters beta and ci may be trained by using a gradient descent method with L2-loss on Y:

L(predY, obsY)=∥predY−obsY∥₂²,

where predY is a predicted probability distribution on deletion lengths (e.g., as computed by the neural network 400 using current parameter values), and obsY is an observed probability distribution on deletion lengths (e.g., based on repair genotype data collected from CRISPR/Cas9 experiments).

Although a neural network is used in the example shown in FIG. 4, it should be appreciated that aspects of the present disclosure are not so limited. For instance, in some embodiments, one or more other types of machine learning techniques, such as linear regression, non-linear regression, random-forest regression, etc., may be used additionally or alternatively.

Furthermore, in some embodiments, one or more neural networks that are different from the neural network 400 may be used additionally or alternatively. As one example, a different activation function may be used for one or more nodes, such as sigma(x)=max(0, x) (rectified linear unit, or ReLU), sigma(x)=0.5*(tanh(x)+1.0) (Sigmoid), sigma(x)=max(0, x)+min(0, x)*0.5*(tanh(x)+1) (Swish), etc. As another example, batch normalization may be performed at one or more hidden layers. As another example, deletion length may be modeled explicitly as an input to a neural network. For instance, there may be three features, deletion length, microhomology length, and GC fraction. The neural network may be trained on L2-loss (sometimes termed “mean-squared error” loss) between predicted frequencies of deletion lengths and observed frequencies of deletion lengths. Any other suitable loss function may be used instead of the L2-loss function including, for example, mean-squared logarithmic error, mean-absolute error or L1-loss, Kullback Leibler (KL) divergence, cross entropy, multi-class cross entropy, negative logarithmic likelihood, Poisson, and Hinge loss.

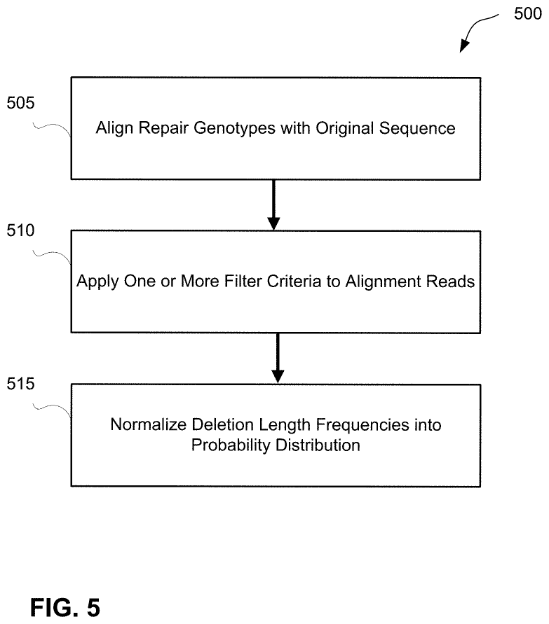

FIG. 5 shows an illustrative process 500 for processing data collected from CRISPR/Cas9 experiments, in accordance with some embodiments. For instance, the process 500 may be performed for each input DNA sequence and CRISPR/Cas9 cut site, and a resulting dataset may be used to train the illustrative neural network 400 of FIG. 4.

At act 505, repair genotypes observed from CRISPR/Cas 9 experiments may be aligned with an original DNA sequence. Any suitable technique may be used to observe the repair genotypes, such as Illumina DNA sequencing. Any suitable alignment algorithm may be used for alignment, such as a Smith-Waterman algorithm or a Needleman-Wunsch algorithm with some suitable scoring parameters (e.g., +1 for match, −2 for mismatch, −4 for gap open, and −1 for gap extend).

At act 510, one or more filter criteria may be applied to alignment reads from act 505. For instance, in some embodiments, only those reads that include a single deletion of length 3 or greater are considered. This may filter out deletions that are unlikely to have resulted from MMEJ. Additionally, or alternatively, only those reads in which a deletion includes at least one base directly 5′ or 3′ of the CRISPR/Cas9 cut site are considered. This may filter out deletions that are unlikely to have resulted from CRISPR/Cas9.

At act 515, frequencies of deletion lengths of interest (e.g., from 3 to 26) may be normalized into a probability distribution.

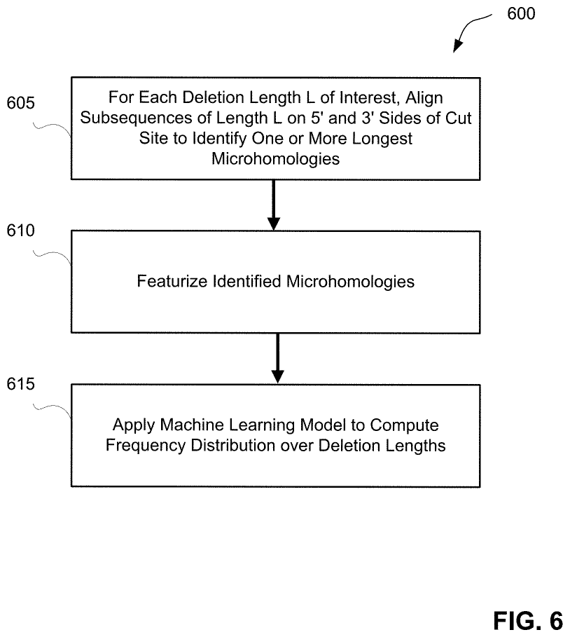

FIG. 6 shows an illustrative process 600 for using a machine learning model to predict frequencies of deletion lengths, in accordance with some embodiments. Acts 605 and 610 may be similar to, respectively, acts 305 and 310 of the illustrative process 300 of FIG. 3, except that acts 605 and 610 may be performed for an input DNA sequence seq and a cut site location for which repair genotype data from an CRISPR/Cas9 experiment may not be available. At act 615, a machine learning model, such as the machine learning model trained at act 315 of the illustrative process 300 of FIG. 3, may be applied to an output of act 610 to compute a frequency distribution over deletion lengths of interest.

The inventors have recognized and appreciated that multiple repair genotypes may be possible for a single deletion length. In some embodiments, given a microhomology match[L][i:j] for a deletion length L, a repair genotype may be constructed by concatenating left[L][−inf:j] with right[L][j:+inf]. With reference to the example shown in FIGS. 2A-D, the repair genotype is simply the overhang 200A (i.e., left[5]), because the microhomology 205 is at the 3′ end of the overhang 200A.

For a deletion length L, suppose M microhomologies are present in match[L]. Given an index m between 1 and M, let sₘ and eₘ denote, respectively, the start index and the end index of the m^{th} microhomology, so that a length of the m^{th} microhomology may be calculated as eₘ−sₘ+1. Furthermore, let RG [L][m] denote the repair genotype of the m^{th} microhomology, as constructed above. In some embodiments, a frequency of occurrence of RG [L][m] may be determined as follows:

$$frequency ( RG [ L ] [ m ] ) = Y [ L ] * \frac{{e}_{m} - {s}_{m} + 1}{{\sum }_{n = 1}^{M} ( {e}_{n} - {s}_{n} + 1 )} ,$$

where frequency Y[L] of deletion length L may be determined in any suitable manner, for example, as discussed above in connection with FIG. 6. In this manner, a repair genotype corresponding to a longer microhomology may be predicted to occur more frequently than a repair genotype corresponding to a shorter microhomology, where the frequencies may be proportional to the respective microhomology lengths.

It should be appreciated that aspects of the present disclosure are not limited to the use of any particular technique for predicting frequencies of repair genotypes. For instance, in some embodiments, a machine learning model may be used to determine frequencies of repair genotypes from frequencies of deletion lengths, in addition to, or instead of, the illustrative function frequency(RG[L][m]) described above.

In some embodiments, one or more of the techniques described herein with respect to 3-26 base pair deletions may be used for other deletion lengths of interest, such as 1-2 base pair deletions. For instance, the illustrative function frequency(RG[L][m]) described above may be used to determine frequencies of repair genotypes from frequencies of deletion lengths of 1-2 base pairs. The frequencies of deletion lengths of 1-2 base pairs may be predicted in any suitable manner, such as using one or more of the techniques described herein with respect to 3-26 base pairs.

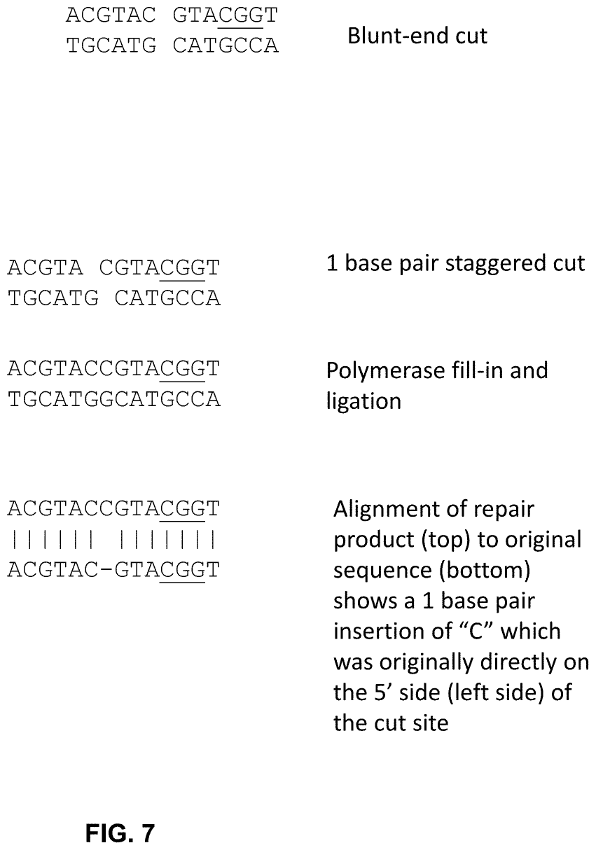

In some embodiments, given an input sequence seq and an insertion frequency Y, the nucleotide seq[−1] may be predicted to be inserted with frequency Y. The inventors have recognized and appreciated that, while Cas9 is typically understood to induce a blunt-end double-strand break, some evidence suggests that Cas9 may generate a 1 base pair staggered end cut instead. FIG. 7 shows illustrative examples of a blunt-end cut and a staggered cut, in accordance with some embodiments.

As discussed above, the inventors have recognized and appreciated at least two tasks of interest: predicting frequencies of deletion lengths, as well as predicting frequencies of repair genotypes. In some embodiments, a single machine learning model may be provided that performs both tasks.

In some embodiments, repair genotypes corresponding to a deletion of length L may be labeled as follows: for every integer K ranging from 0 to L, a K-genotype associated with deletion length L may be obtained by concatenating left[L][−inf:K] with right[L][K:+inf]. A vector COLLECTION of length Q where each element is a tuple (K, L) may be constructed by enumerating each K-genotype for each deletion length L of interest and removing tuples that have the same repair genotype, e.g., (k′, L) and (k, L) such that left[L][−inf:k′] concatenated with right[L][k′:+inf] is equivalent to left[L][−inf:k] concatenated with right[L][k:+inf], for example, by retaining only the tuple with the larger K. A training data set may be constructed using observational data by constructing a vector X of length Q where X sums to 1 and X[q] represents an observed frequency of a repair genotype generated by COLLECTION[q].

In some embodiments, the vector COLLECTION may be featurized. This may be performed for a given tuple (k, l) by determining whether there is an index i such that match[l][i:k] is a microhomology. If no such i exists, then the tuple (k, l) may be considered to not partake in microhomology.

The inventors have recognized and appreciated that frequencies of repair products may be influenced by certain features of microhomologies such as microhomology length, fraction of G-C pairings, and/or deletion length. The inventors have also recognized and appreciated that some default values may be useful for repair genotypes that are considered to not partake in microhomology.

For example, the inventors have recognized and appreciated that energetic stability of a microhomology may increase proportionately with a length of the microhomology. Accordingly, in some embodiments, the microhomology length k−i may be used for a tuple (k, l), and a default value of 0 may be used if (k, l) does not partake in microhomology.

As another example, the inventors have recognized and appreciated that thermodynamic stability of a microhomology may depend on specific base pairings, and that G-C pairings have three hydrogen bonds and therefore have higher thermodynamic stability than A-T pairings, which have two hydrogen bonds. Accordingly, in some embodiments, a GC fraction, as shown below, may be used as a feature for (k, l), where indicator (boolean) equals 1 if boolean is true, and 0 otherwise. A default value of −1 may be used if (k, l) does not partake in microhomology.

$$\frac{{\sum }_{j = 1}^{k - 1} indicator ( left [ l ] [ j ] = ' G ' or ' C ' )}{k - i}$$

In some embodiments, a feature for deletion length may be considered, represented as l for the tuple (k, l). The inventors have also recognized and appreciated (e.g., from experimental data) that 0-genotype and l-genotype repair products may occur despite a lack of microhomology, and may occur through microhomology-free end-joining repair pathways. Accordingly, (k, l) may be featurized with a Boolean for 0-genotype that is equal to 1 if k=0 and (k, l) does not partake in microhomology, and 0 otherwise. A Boolean feature for l-genotypes may also be used where it is equal to 1 if k=l and (k, l) does not partake in microhomology, and 0 otherwise.

In some embodiments, Z may be normalized into a probability distribution over all unique repair genotypes of interest within all deletion lengths of interest (e.g., deletion lengths between 3 and 26). The inventors have recognized and appreciated (e.g., from experimental data) that frequency may decrease exponentially with deletion length. Accordingly, in some embodiments, an exponential linear model may be used to normalize the vector of repair genotype scores. For example, the following formula may be used:

$$Y [ q ] = \frac{exp ( Z [ q ] - beta * DL [ q ] )}{{\sum }_{{q}^{′} = 1}^{Q} exp ( Z [ {q}^{′} ] - beta * DL [ {q}^{′} ] )}$$

where DL[q]=l for each q where COLLECTIONS[q]=(k, l), and beta is a parameter.

In some embodiments, a probability distribution Y over all unique repair genotypes of interest within all deletion lengths of interest may be converted to a probability distribution Y′ over all deletion lengths. The following formula may be used for this:

$${Y}^{′} [ l ] = \frac{{\sum }_{q = 1}^{Q} Y [ q ] * indicator ( DL [ q ] = l )}{{\sum }_{q = 1}^{Q} Y [ q ]}$$

In some embodiments, the parameter beta may be initialized to −1. These parameters may then be trained using repair genotype data collected from CRISPR/Cas9 experiments.

In some embodiments, the parameters w [h] and b[h] for each hidden layer h and the parameters beta may be trained by using a gradient descent method with L2-loss on Y:

L(predY, obsY)=||predY−obsY||₂²,

where predY is a predicted probability distribution on deletion lengths (e.g., as computed by the neural network 400 of FIG. 4 using current parameter values), and obsY is an observed probability distribution on deletion lengths (e.g., based on repair genotype data collected from CRISPR/Cas9 experiments).

In other embodiments, MMEJ deletion lengths may be predicted using the following formula:

$$Pattern Score = \frac{exp ( {aM}^{*} )}{{Δ}^{b ( M )}}$$

$$M = Matches ( GC = 2 , AT = 1 )$$

$${M}^{*} = Matches ( GC = 2 , AT = 1 , G = 0.5 )$$

$$Δ = Deletion Length$$

$$a = Match Parameter$$

$$b = Deletion Length Parameter .$$

The inventors have recognized and appreciated that one or more of the techniques described herein may be used to identify therapeutic guide RNAs that are expected to produce a therapeutic outcome when used in combination with a genomic editing system without an HDR template. For instance, one or more of the techniques described herein may be used to identify a therapeutic guide RNA that is expected to result in a substantial fraction of genotypic consequences that cause a gain-of-function mutation in DNA in the absence of an HDR template. A therapeutic guide RNA may be used singly, or in combination with other therapeutic guide RNAs. An action of the therapeutic guide RNA may be independent of, or dependent on, one or more genomic consequences of the other therapeutic guide RNAs.

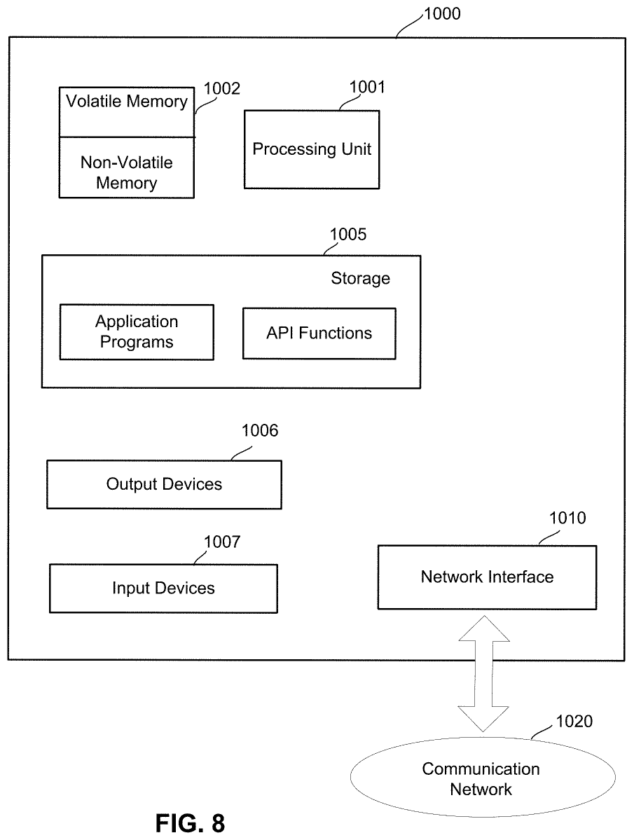

FIG. 8 shows, schematically, an illustrative computer 1000 on which any aspect of the present disclosure may be implemented. In the embodiment shown in FIG. 8, the computer 1000 includes a processing unit 1001 having one or more processors and a non-transitory computer-readable storage medium 1002 that may include, for example, volatile and/or non-volatile memory. The memory 1002 may store one or more instructions to program the processing unit 1001 to perform any of the functions described herein. The computer 1000 may also include other types of non-transitory computer-readable medium, such as storage 1005 (e.g., one or more disk drives) in addition to the system memory 1002. The storage 1005 may also store one or more application programs and/or external components used by application programs (e.g., software libraries), which may be loaded into the memory 1002.

The computer 1000 may have one or more input devices and/or output devices, such as devices 1006 and 1007 illustrated in FIG. 8. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, the input devices 1007 may include a microphone for capturing audio signals, and the output devices 1006 may include a display screen for visually rendering, and/or a speaker for audibly rendering, recognized text.

As shown in FIG. 8, the computer 1000 may also comprise one or more network interfaces (e.g., the network interface 1010) to enable communication via various networks (e.g., the network 1020). Examples of networks include a local area network or a wide area network, such as an enterprise network or the Internet. Such networks may be based on any suitable technology and may operate according to any suitable protocol and may include wireless networks, wired networks or fiber optic networks.

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

In certain embodiments, the machine learning algorithm is defined by the following code:

```
sequences = open(‘Spacer2Sequence.txt’,‘r’)
### Split sequences by CRISPR Cas9 cut site
split_seq = { }
for line in sequences:
    line = line.strip(‘\n’)
    full = line.split( )
    spacer = int(full[0])
    seq = full[2]
    cut_site = int(full[1]) + 1
    split_seq[spacer] = [seq[:cut_site], seq[cut_site:]]
#for id in sorted(split_seq):
```

| | |
|---|---|
| # | print str(id) + ‘\t’ + str(split_seq[id][0]) + ‘\t’ + str(split_seq[id][1]) |

```
### Import aggregated distributions
agg_dist =
open(‘Aggregated_Deletion_Length_Distributions_Total.txt’,‘r’)
agg_dist_data = { }
for line in agg_dist:
    line = line.strip(‘\n’)
    full = line.split( )
    spacer = int(full[0])
    agg_dist_data[spacer] = full[1:]
### Function for microhomology
def microhomology(a,b):
    matches = [ ]
    consecutive_matches = [ ]
    temp = 0
    good_matches = [ ]
    for seq1,seq2 in zip(a,b):
        if seq1 == seq2 or seq1 == ‘G’ or seq2 == ‘C’:
            if seq1 == seq2:
                if seq1 == ‘G’ or seq1 == ‘C’:
                    matches.append(2.5)
                else:
                    matches.append(1.5)
                if len(a) == 1:
                    matches.append(0.5)
            elif seq1 == ‘G’ or seq2 == ‘C’:
                matches.append(1)
        else:
            matches.append(0)
    if sum(matches) == 0:
        consecutive_matches.append(0)
    else:
        for score in matches:
            if score > 0:
                temp += score
            else:
                consecutive_matches.append(temp)
                temp = 0
        consecutive_matches.append(temp)
```

| | |
|---|---|
| # | for m in consecutive_matches: |
| # | if m >= 3: |
| # | good_matches.append(m) |
| # | if len(good_matches) >= 1: |
| # | max_cons_matches = sum(good_matches) |
| # | else: |
| # | max_cons_matches = max(consecutive_matches) |

```
    max_cons_matches = max(consecutive_matches)
    mhs = sum(i >= 3 for i in consecutive_matches)
    return [max_cons_matches, mhs]
def scorer(s1, s2):
# Input: Two sequences, across a deletion
MATCH = {‘G’: 2.5, ‘C’: 2.5, ‘A’: 1.5, ‘T’: 1.5}
PROMISCUOUS_G = 1
# SINGLE_BASE_AT = 0.5
SCOREINIT = math.exp(−5)
scores = [ ]
curr_score = SCOREINIT # prevent 0's
for i in range(len(s1)):
  if s1[i] == S2[i]:
  curr_score += MATCH[s1[i]]
  else:
  if s1[i] == ‘G’ or S2[i] == ‘C’:
  curr_score += PROMISCUOUS_G
  else:
  # break
  scores.append(curr_score)
  curr_score = SCOREINIT
scores.append(curr_score)
return max(scores)
### MMEJ deletion distribution predictions
import math
def power(x):
```

| | |
|---|---|
| # | power = 0.45*x + 0.7475 |

```
    power = 0.485*x + 0.7772
    return power
#def dist_coeff(y):
```

| | |
|---|---|
| # | coeff1 = 0.054*math.exp(0.5555*y) |
| # | return coeff1 |

```
dist_pred = { }
calc_dist_power = { }
mhs_del = { }
for spacer in split_seq:
    seq1 = split_seq[spacer][0]
    seq2 = split_seq[spacer][1]
    for del_1 in range(1,31):
        if spacer not in dist_pred:
            dist_pred[spacer] = [0]*30
        seq1_new = seq1[−del_1:]
        seq2_new = seq2[:del_1]
        mh_score = microhomology(seq1_new, seq2_new)
        my_mh = scorer(seq1_new, seq2_new)
        if mh_score[0] != my_mh − math.exp(−5):
            print mh_score[0], my_mh − math.exp(−5)
            print seq1_new, seq2_new, ‘\n’
        dist_power = power(mh_score[0])
```

| | |
|---|---|
| # | dist_c1 = dist_coeff(mh_score[0]) |

```
        dist_pred[spacer][del_1−1] =
math.exp(float(1.8*mh_score[0]))/floa(float(del_1)**dist_power)
        if mh_score[0] not in calc_dist_power:
            calc_dist_power[mh_score[0]] = [[ ],[ ]]
        calc_dist_power[mh_score[0]][0].append(del_1)
        calc_dist_power[mh_score[0]][1].-
        append(agg_dist_data[spacer][del_1−1])
        if spacer not in mhs_del:
            mhs_del[spacer] = [0]*30
        mhs_del[spacer][del_1−1] = mh_score[1]
### Output predicted distributions
for spacer in dist_pred:
    norm = [float(i)/sum(dist_pred[spacer]) for i in dist_pred[spacer]]
    # print str(spacer) + ‘\t’ + ‘\t’.join(map(str, norm))
### Output data to create distance power function
#for match in sorted(calc_dist_power):
```

| | |
|---|---|
| # | for i in range(0, len(calc_dist_power[match][0])): |
| # | print str(match) + ‘\t’ + str(calc_dist_power[match][0][i]) + |

‘\t’ + str(calc_dist_power[match][1][i])
### Output number of microhomologies for given deletion length
#for spacer in mhs_del:

| | |
|---|---|
| # | print str(spacer) + ‘\t’ + ‘\t’.join(map(str, mhs_del[spacer])) |

1. A method for selecting one or more guide RNAs (gRNAs) from a plurality of gRNAs for CRISPR, comprising acts of:
for at least one gRNA of the plurality of gRNAs, using a local DNA sequence and a cut site targeted by the at least one gRNA to predict a frequency of one or more repair genotypes resulting from template-free repair of the local DNA sequence following application of CRISPR to the local DNA sequence with the at least one gRNA; and
selecting the at least one gRNA based at least in part on the predicted frequency of the one or more repair genotypes.

2. The method of claim 1, wherein the one or more repair genotypes correspond to one or more healthy alleles of a gene related to a disease.

3. The method of claim 1, wherein the predicted frequency of the one or more repair genotypes is at least about 50%.

4. The method of claim 1, wherein predicting the frequency of the one or more repair genotypes comprises:
for each deletion length of a plurality of deletion lengths, aligning subsequences of that deletion length on 5′ and 3′ sides of the cut site to identify one or more longest microhomologies;
featurizing the identified microhomologies;
applying a machine learning model to compute a frequency distribution over the plurality of deletion lengths, wherein the identified microhomologies each comprise a number of matching bases, wherein the computation includes a non-linear function of the number of matching bases in the identified microhomologies; and
using the frequency distribution over the plurality of deletion lengths to determine the frequency of the one or more repair genotypes.

5. The method of claim 4, wherein featurizing the identified microhomologies comprises determining a G-C fraction value for each of the identified microhomologies.

6. The method of claim 5, wherein featurizing the identified microhomologies further comprises determining a microhomology length of each of the identified microhomologies.

7. The method of claim 4, wherein applying the machine learning model comprises applying a neural network model.

8. The method of claim 1, wherein predicting the frequency of the one or more repair genotypes comprises:
for each deletion length of a plurality of deletion lengths, aligning subsequences of that deletion length on 5′ and 3′ sides of the cut site to identify one or more longest microhomologies;
determining feature values for the identified microhomologies; and
providing the feature values as input to a machine learning model to obtain output indicating a probability distribution over a plurality of deletion lengths.

9. The method of claim 8, wherein predicting the frequency of the one or more repair genotypes further comprises:
using the probability distribution over the plurality of deletion lengths to determine the frequency of the one or more repair genotypes.

10. The method of claim 1, wherein the plurality of gRNAs comprise gRNAs for CRISPR/Cas9, and the application of CRISPR comprises application of CRISPR/Cas9.

11. A system comprising:
at least one processor; and
at least one computer-readable storage medium having encoded thereon instructions which, when executed, cause the at least one processor to perform the method of claim 1.

12. At least one computer-readable storage medium having encoded thereon instructions which, when executed, cause at least one processor to perform the method of claim 1.

13. A method for CRISPR editing of DNA that utilizes a guide RNA in the absence of a homology directed repair template, the method comprising selecting the guide RNA to produce one or more selected genotypic outcomes.

14. A method of predicting a frequency of one or more repair genotypes resulting from template-free repair following application of template-free CRISPR/Cas to a target nucleotide sequence, the method comprising:
using at least one computer hardware processor to perform:
for each deletion length of a plurality of deletion lengths, aligning subsequences of that deletion length on 5′ and 3′ sides of a cut site to identify one or more longest microhomologies;
determining feature values for the identified microhomologies;
providing the feature values as input to a machine learning model to obtain output indicating a probability distribution over the plurality of deletion lengths; and
using the probability distribution over the plurality of deletion lengths to determine the frequency of the one or more repair genotypes.

15. The method of claim 14, wherein determining the feature values comprises:
determining a G-C fraction value for each of the identified microhomologies.

16. The method of claim 14, wherein determining the feature values comprises:
determining a microhomology length of each of the identified microhomologies.

17. The method of claim 14, wherein the machine learning model comprises a neural network model.

18. The method of claim 17, wherein the neural network model comprises multiple hidden layers.

19. The method of claim 17, comprising:
for each deletion length of the plurality of deletion lengths, aligning subsequences of that deletion length on 5′ and 3′ sides of the cut site to identify two or more longest microhomologies.

20. A system comprising:
at least one processor; and
at least one computer-readable storage medium having encoded thereon instructions which, when executed, cause the at least one processor to perform the method of claim 14.

21. At least one computer-readable storage medium having encoded thereon instructions which, when executed, cause at least one processor to perform the method of claim 14.
